(19) United States
(12) Patent Application Publication
(10) Pub. No.: US 2013/0136811 A1
(43) Pub. Date: May 30, 2013

(54) Compositions and Methods for Pain Reduction

(71) Applicant: Steven C. Schachter, Sharon, MA (US)

(72) Inventor: Steven C. Schachter, Sharon, MA (US)

(73) Assignee: PRESIDENT AND FELLOWS OF HARVARD COLLEGE, Cambridge, MA (US)

(21) Appl. No.: 13/699,362

(22) PCT Filed: May 26, 2011
(86) PCT No.: PCT/US11/38089

§ 371 Date: Feb. 15, 2013

Related U.S. Application Data
(60) Provisional application No. 61/348,381, filed on May 26, 2010.

(51) Int. Cl. A61K 36/725; A61K 31/352; A61K 36/65; A61K 36/539; A61K 36/484

(52) U.S. Cl. CPC A61K 36/725; A61K 36/539; A61K 36/65; A61K 31/352
USPC 424/741; 514/456; 549/403

(57) ABSTRACT

Compositions containing four-herb extract combination or Wogonin are useful in methods for the reduction of pain. A four-herb combination is used to treat pain as well as reduce or prevent epileptic seizures.

### BACKGROUND OF THE INVENTION

Pain management remains a ubiquitous clinical problem. In addition to injury, nearly every disease or pathological condition from arthritis to cancer to HIV infection and diabetes has a major pain component. Pain management for some conditions such as nerve injuries and chronic inflammatory disease has been poor. While a number of drugs exist to alleviate pain, the use of many of them is limited by safety issues and side effects.

### SUMMARY OF THE INVENTION

The invention features compositions and methods for the reduction of pain, e.g., neuropathic pain or inflammatory pain by administering to a subject a formulation comprising a purified combination of herbs comprising Scutellaria, Glycyrrhiza, Ziziphus and Paeonia. For example, the formulation is a pharmaceutical composition that contains active ingredients, a combination of extracts or compounds purified from each herb, and optionally contains pharmaceutically-acceptable excipients or carriers. The composition comprises two, three, or four of the listed herbs or compounds puried from two, three, or all four of the four listed herbal sources.

The pharmaceutical composition comprises or consists essentially of, or consists of processed plant material of a combination of herbs: Scutellaria, Glycyrrhiza, Ziziphus and Paeonia. A method of preventing and reducing pain perception is carried out by administering to a subject the combination of plant extracts in an amount to reduce pain perception by at least 10% compared to the level of pain perception in the absence of the combination herb composition. Preferably, pain is reduced by 25%, 50%, 2-fold, 5-fold, 10-fold or more. More preferably, pain is eliminated. The composition comprises an extract or purified compound from two, three or all four of the listed herbs. For example, wogonin is a compound purified from the herb, Scutellaria. In another example, purified Wogonin is optionally combined with or administered together with one, two, three, or all four of the listed herbs. The combination of four herbs of the present invention provides a synergistic effect with respect to potency and prolonging of the duration of action in treating or reducing pain, such as neuropathic pain or inflammatory pain in a subject.

The combination herb composition, e.g. 4-herb combination, is also useful to prevent the onset of and reduce the severity of a seizure disorder such as epilepsy. For example, the frequency, duration, or severity of seizures is reduced by 25%, 50%, 2-fold, 5-fold, 10-fold or more. Seizure frequency, duration and severity are evaluated and monitored by health care providers as key outcomes for the treatment of epilepsy. Frequency refers to the number of seizures over a specified time period. Duration refers to the amount of time that a seizure lasts. Severity refers to the direct impact of an individual seizure on the patient. The desired outcome for the treatment of epilepsy is reduced seizure frequency, shorter seizure duration, and reduced severe severity. The optimal response is complete freedom from seizures. Information about seizure frequency, duration and severity is related to physicians by patients or others who have witnessed the patients' seizures. The information may be related verbally, e.g., during office visits, or in written form, such as diaries, that is given to health care providers during office visits or sent to them by mail or electronic means. The combination of four herbs provides a significant synergistic effect for treating and reducing seizure disorders, such as epilepsy and/or seizures resulting from traumatic brain injury in a subject.

The present invention also provides compositions and methods for the reduction of pain by administering to a subject a formulation comprising purified Wogonin. Wogonin has been found to be useful to reduce the severity and perception of pain. A purified Wogonin compound or an analogue thereof is used to treat both acute and chronic pain indications. Accordingly, a method of preventing and reducing pain perception is carried out by administering to a mammal a composition comprising purified Wogonin in an amount sufficient to reduce pain perception by at least 10% compared to the level of pain perception in the absence of the composition. Preferably, pain is reduced by 25%, 50%, 2-fold, 5-fold, 10-fold or more. Reduction in pain is measured using standard medical methods of evaluating and scoring pain, e.g., the McGill Pain Questionnaire.

The compositions are suitable for humans or other mammalian subjects, e.g., the mammal is a human, canine, feline, or equine subject. In one example, the subject to be treated is identified as suffering from neuropathic pain. For example, the subject is identified as suffering from diabetes, HIV infection, neuropathic pain related to chemotherapy, or pain associated with nerve compression such as sciatica or a herniated disc. Alternatively, the subject is identified as suffering from inflammatory pain such as that associated with tissue injury, damage or disease.

Wogonin to be used in the methods described herein is purified, e.g., isolated from natural sources or chemically synthesized. A purified preparation of Wogonin comprises at least 75%, 80%, 90% or 99%-100% Wogonin by weight (w/w). An exemplary dose range for administration of Wogonin is 0.1 mg/kg/day to 1000 mg/kg/day. For example, 500 mg/kg is administered to the patient. The compositions are administered locally or systemically to alleviate the perception of pain. For example, the composition is administered in a sustained release delivery vehicle or in the form of an adhesive dermal patch. Alternatively the composition is continuously infused into the subject, administered by an intravenous pump, or orally ingested. In some embodiments, the composition comprises an implant, e.g., a biodegradable or erodible implant, or a semipermeable membrane, e.g., a delivery rate controlling membrane. In other formulations, the composition comprises a plurality of particles, where each of the particles is characterized by a different rate of dissolution. Standard formulations such as an ointment, paste, spray, patch, cream, gel, sponge, foam, or subcutaneous depo formulation are also encompassed by the invention.

Major categories of pain to be treated include post-operative and post trauma pain; non-malignant chronic pain disorders such as osteoarthritis and rheumatoid arthritis, fibromyalgia, multiple sclerosis and headache; neuropathic pain such as that associated with peripheral nerve damage, diabetes, and Human Immunodeficiency Virus (e.g., HIV-1, AIDS) infection; back pain such as that associated with disc avulsion or nerve compression; and cancer pain, including pain secondary to chemotherapy. Neuropathic pain includes chronic pain resulting from injury to the nervous system, e.g., an injury to the central nervous system (brain and spinal cord) or the peripheral nervous system (nerves outside the brain and spinal cord). In some cases, neuropathic pain occurs after trauma and is associated with pathologic conditions such as multiple sclerosis and stroke. Neuropathic pain is also associated with shingles (post-herpetic neuralgia due to Varicella-zoster virus). Neuropathic pain does not respond well to conventional pain remedies, e.g., opiate drugs such as morphine. Wogonin can be used to alleviate, suppress or inhibit the existing pain, as well as prevent pain from arising from a pain-causing event or disorder.

A method of reducing pain perception is characterized by identifying a subject with an injury and administering to the individual an amount of Wogonin sufficient to reduce pain perception by at least 10% compared to the level of pain perception in the absence of a medicament. Preferably, perception of pain is reduced by at least 20%, 50%, 75%, eliminated or rendered imperceptible by the patient. In one example, the subject has been diagnosed with an injury to a bodily tissue or inflammation of a bodily tissue. For example, the injury is a cut/incision, abrasion, bruise, fracture, crush injury or is the result of a surgical procedure. The subject is identified as experiencing pain related to passage of kidney stones, a dental extraction, caesarian surgery, or cancer. For example, administration for pain is carried out using a sustained release formulation.

Alternatively, the subject is identified as experiencing pain in the absence of an injury or inflammation of a bodily tissue. Purified Wogonin is administered to the individual as described above in an amount sufficient to reduce pain perception by at least 10% compared to the level of pain perception in the absence of a medicament. Examples of such pain syndromes include patients identified as suffering from neuropathic pain, e.g., neuropathic pain associated with diabetes or HIV infection.

Based on the data showing a neuroprotective effect of Wogonin, Wogonin confers clinical benefit to individuals afflicted with other pathological disorders that can result in neuropathic pain. For example, a subject is identified as suffering from or at risk of developing post-herpetic neuralgia or Reflex Sympathetic Dystrophy Syndrome (RSD)/Complex Regional Pain Syndrome (CRPS). The latter syndrome is a chronic neurological syndrome characterized by one or more of the following symptoms: severe burning pain, pathological changes in bone and skin, excessive sweating, tissue swelling, or extreme sensitivity to touch.

Wogonin is administered alone or in combination with a second or third pain (or more) medication(s). Wogonin is administered at a dose to reduce pain by at least 10% with few or no side effects. For example, the Wogonin prevents the development of or completely eliminates pain. The dose preferably does not exceed 1000 mg/kg of body weight/day. For example, the dose does not exceed 500, 400, 300, 250, 100, 50, or 10 mg/kg/day. Mode of administration is oral, intravenous, subcutaneous, or topical. Topical administration, e.g., in the form of a cream, foam, or ointment, is useful to alleviate neuropathic pain, e.g., pain associated with shingles.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned 125 herein are incorporated by reference in their entirety. In the case of conflict, the present specification, including definitions, will control. In addition, the materials, methods, and examples are illustrative only and not intended to be limiting.

### BRIEF DESCRIPTION OF THE DRAWINGS

FIG. 1 is a diagram of the chemical structure of Wogonin.

FIG. 2 is a line graph showing the effect of Wogonin on neuroprotective effect of Wogonin against NMDAR-mediated excitotoxicity in primary neuronal cultures from mouse cortex.

FIG. 3 is flow chart showing the steps of a purification method for Wogonin.

FIG. 4 is spectrophotometic scan of purified Wogonin. ¹H-NMR (400 MHz), ¹³C-NMR (100MHz) & DEPT spectra of Wogonin (compound 3) from the root of Scutellaria baicalensis.

FIG. 5 shows the combinations of two or three of the listed 4 herbs in this invention. A. The combination of two of the 4 herbs listed in this invention. “x” indicates the available combinations. B. The combination of three of the 4 herbs listed in this invention. “x” indicates 140 selected herbs.

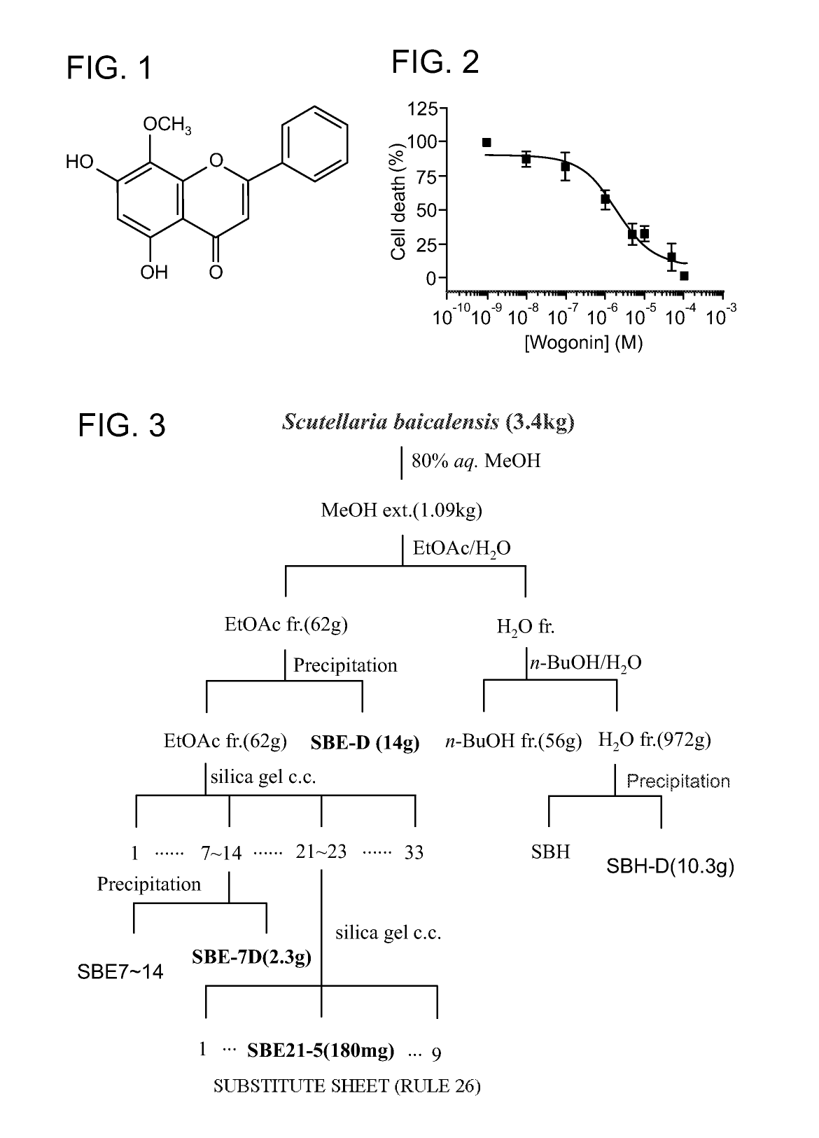

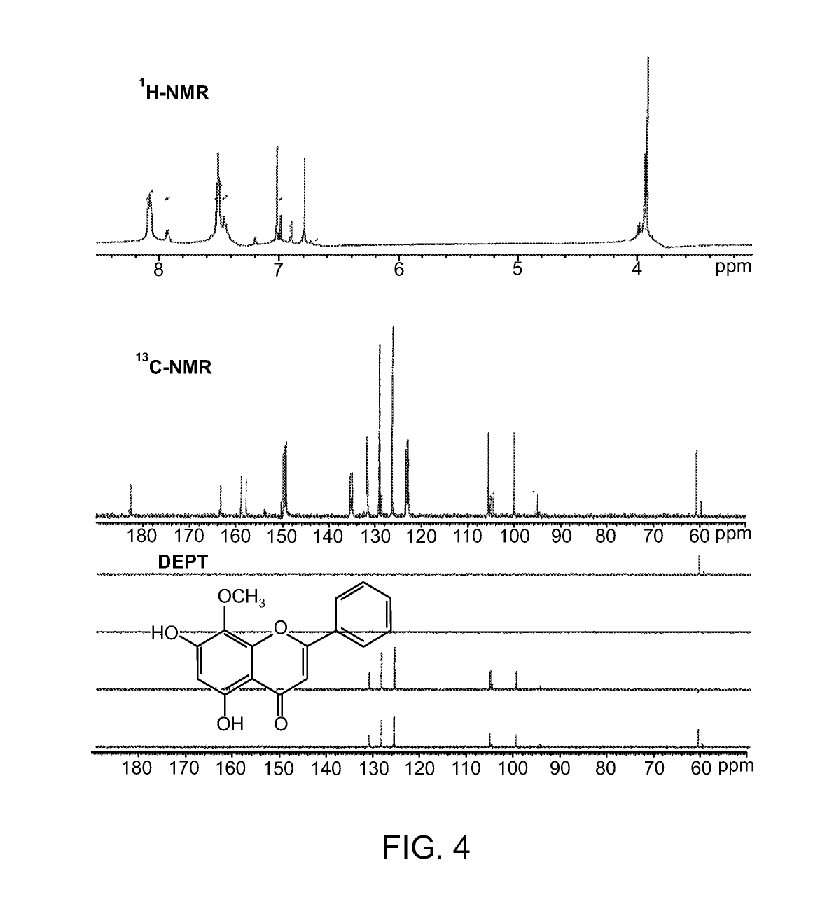

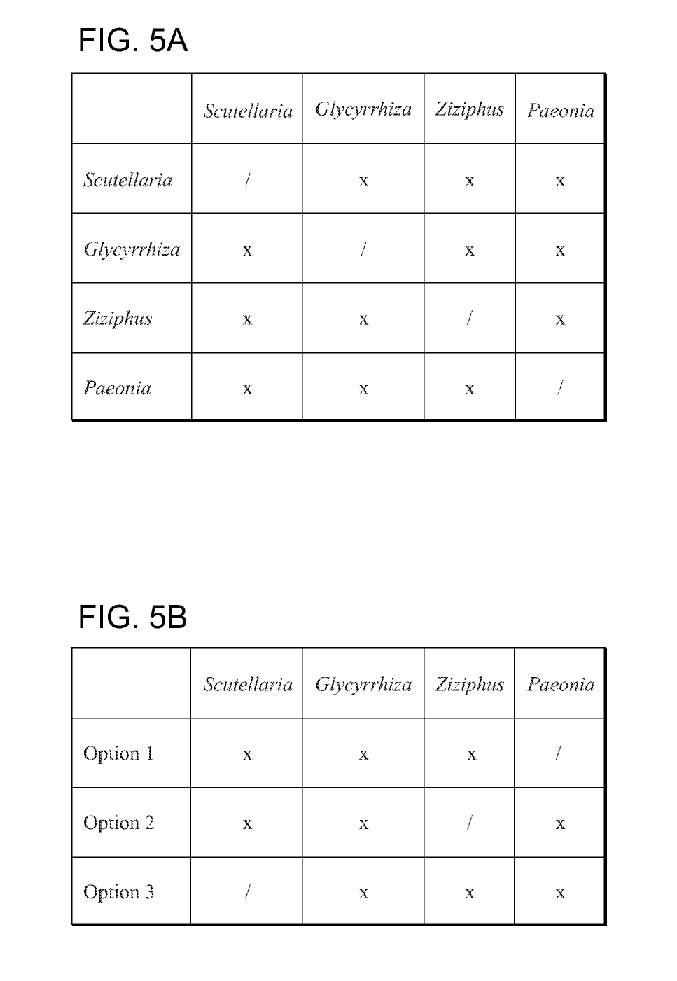

### DETAILED DESCRIPTION

The invention provides methods and compositions for treating or alleviating pain using a combination of compounds purified or isolated from natural products such as botanical or herbal extracts. Such compounds include, for example, compounds or extract purified from herbs Scutellaria, Glycyrrhiza, Ziziphus and Paeonia, in optically active or racemic form, salts, and hydrates thereof.

As used herein, the terms “isolated”, “purified” and “substantially purified,” are used interchangeably and refer to a compound that is at least 30%, 40%, or preferably 50%, by weight, free from proteins and naturally-occurring organic molecules with which it is naturally associated. More preferably, the preparation is at least 60%, more preferably 75%, more preferably 90%, and most preferably at least 99%-100%, by weight, chemical compound, e.g., natural product compound. A purified compound may be obtained by any method known in the art or described herein including, for example, high pressure liquid chromatography, thin layer chromatography, or by synthesis. A combination of four-herb and Wogonin are useful for the treatment of pain.

“Pain” is an unpleasant sensory and emotional experience. Pain classifications have been based on duration, etiology or pathophysiology, mechanism, intensity, and symptoms. The term “pain” as used herein refers to all categories of pain, including pain that is described in terms of stimulus or nerve response, e.g., somatic pain (normal nerve response to a noxious stimulus) and neuropathic pain (abnormal response of a injured or altered sensory pathway, often without clear noxious input); pain that is categorized temporally, e.g., chronic pain and acute pain; pain that is categorized in terms of its severity, e.g., mild, moderate, or severe; and pain that is a symptom or a result of a disease state or syndrome, e.g., inflammatory pain, cancer pain, AIDS pain, arthropathy, migraine, trigeminal neuralgia, cardiac ischaemia, and diabetic peripheral neuropathic pain (see, e.g., Harrison's Principles of Internal Medicine, pp. 93-98 (Wilson et al., eds., 12th ed. 1991); Williams et al., J. of Med. Chem. 42: 1481-1485 (1999), herein each incorporated by reference in their entirety). “Pain” is also meant to include mixed etiology pain, dual mechanism pain, allodynia, causalgia, central pain, hyperesthesia, hyperpathia, dysesthesia, and hyperalgesia.

Major categories of pain to be treated include post-operative and post trauma pain; non-malignant chronic pain disorders such as osteoarthritis and rheumatoid arthritis, fibromyalgia, multiple sclerosis and headache; neuropathic pain such as that associated with peripheral nerve damage, diabetes, and Human Immunodeficiency Virus (e.g., HIV-1, AIDS) infection; back pain such as that associated with disc avulsion or nerve compression; and cancer pain, including pain secondary to chemotherapy. Neuropathic pain includes chronic pain resulting from injury to the nervous system, e.g., an injury to the central nervous system (brain and spinal cord) or the peripheral nervous system (nerves outside the brain and spinal cord). In some cases, neuropathic pain occurs after trauma and is associated with pathologic conditions such as multiple sclerosis and stroke. Neuropathic pain is also associated with shingles (post-herpetic neuralgia due to Varicella-zoster virus). Neuropathic pain does not respond well to conventional pain remedies, e.g., opiate drugs such as morphine. A combination of four herbs comprising Scutellaria, Glycyrrhiza, Ziziphus and Paeonia and/or Wogonin can be used to alleviate, suppress or inhibit the existing pain, as well as prevent pain from arising from a pain-causing event or disorder.

The term “treatment” or “treating” refers to a decrease in the symptoms associated with the disorder or an amelioration of the recurrence of the symptoms of the disorder, prophylaxis, or reversal of a disease or disorder, or at least one discernible symptom thereof. The term “treatment” or “treating” refers to inhibiting or slowing the progression of a pain or seizures. These terms also refer to suppressing, reducing or inhibiting pain, such as neuropathic pain, inflammatory pain, or pain due to tissue injury as well as seizure disorders. These terms apply to acute or chronic pain.

The term “prevention” or “preventing” refers to delaying the onset of the symptoms of pain or seizures.

The term “neuroprotection” or “neuroprotective activity” as used herein refers to protection against development of pain such as neuropathic pain. The terms also refer to protection from other neurological disorders such as seizure disorders, e.g., epileptic seizures.

The phrase “pharmaceutically acceptable salt(s),” as used herein includes, but is not limited to, salts of acidic or basic groups that may be present in the natural product compounds, and hydrates thereof. Natural product compounds, and hydrates thereof that are basic in nature are capable of forming a wide variety of salts with various inorganic and organic acids. The acids that may be used to prepare pharmaceutically acceptable salts of such basic compounds are those that form salts comprising pharmacologically acceptable anions including, but not limited to, acetate, benzenesulfonate, benzoate, bicarbonate, bitartrate, edetate, camsylate, carbonate, bromide, chloride, iodide, citrate, dihydrochloride, edisylate, estolate, esylate, fumarate, gluceptate, gluconate, glutamate, glycollylarsanilate, hexylresorcinate, hydrabamine, hydroxynaphthoate, isethionate, lactate, lactobionate, malate, maleate, mandelate, mesylate, methylsulfate, muscate, napsylate, nitrate, panthothenate, phosphate/diphosphate, polygalacturonate, salicylate, stearate, succinate, sulfate, tannate, tartrate, teoclate, triethiodide, and pamoate (i.e., 1,1′-methylene-bis-(2-hydroxy-3-naphthoate)). Natural product compounds, and hydrates thereof that include an amino moiety can also form pharmaceutically acceptable salts with various amino acids, in addition to the acids mentioned above. Natural product compounds, and hydrates thereof that are acidic in nature are capable of forming base salts with various pharmacologically acceptable cations. Examples of such salts include alkali metal or alkaline earth metal salts and, particularly, calcium, magnesium, sodium, lithium, zinc, potassium, and iron salts.

The term “subject” as used herein is intended to include a living organism in which alleviation of symptoms or inhibition of a neurological disorder is sought. Preferred subjects are mammals. Examples of subjects include but are not limited to, humans, monkeys, dogs, cats, mice, rats, cows, horses, pigs, goats and sheep.

The term “duration of action” refers herein to the length of time a composition exhibits a desired pharmacologic effect after administration. This is determined by the amount of time drug concentration is at or above the minimum effective concentration. The duration of drug in the body is not equivalent to the duration of effect. A drug may be in the body for a period of time that is much longer than the duration of action, if the concentration remains below the minimum effective concentration. In fact, some drugs that are slowly absorbed may never exert a pharmacologic effect, even though they are in the body for a prolonged period of time. This occurs when the drug is absorbed so slowly that it never reaches concentrations that meet or exceed the minimum effective concentration.

The term “prolonging,” as used herein with respect to the duration of action, refers to the increase in the length of time a composition exhibits a desired pharmacologic effect after administration in combination with a duration-of-action-prolonging agent or composition.

The term “potency” is generally a comparison measure of the relative concentration of a composition required to achieve a given magnitude of response (e.g., anesthesia). This comparison is often made by determining the concentration necessary to produce 50% of the maximal effect (EC.sub.50) for both compounds. The compound with the lower EC50 is the more potent compound. When the concentration response curve for a drug shifts to the right, it is an indication that the potency has decreased. This can happen in disease states where the target organ becomes less responsive to the drug, such that more drug is needed to achieve a given response.

As used herein, the term “extract” refers to a concentrated preparation of a vegetable or animal drug obtained by removing the active constituents therefrom with a suitable menstruum (solvent), evaporating all or nearly all the solvent and adjusting the residual mass or powder to a prescribed standard. Extracts are prepared in three forms, semiliquid or of syrupy consistency, pilular or solid, and as dry powder (see graylab.ac.uk/cgi-bin/omd?query=extract).

As used herein, an “herbal composition or formulation” refers to any composition or formulation which includes herbs, herbal plants, herbal plant parts and/or herbal extracts. Thus, as used herein, an herbal composition or formulation includes herbal preparation comprising herbal food supplements, herbal medicines, herbal drugs, and medical foods. Examples of herbal compositions include, but are not limited to, the following components: a whole plant or a plant part of a single plant species; whole plants or plant parts of multiple plant species; multiple components derived from a single plant species; multiple components derived from multiple plant species; herbal extracts; or any combination of these various components. Also contemplated are herbal compositions comprising one or more chemicals derived from a single or multiple plant species.

Unless otherwise defined, all technical and scientific terms used herein have the same meaning as commonly understood by one of ordinary skill in the art to which this invention belongs.

### Four-Herb Combination for Pain Alleviation and Epilepsy

The present invention provides compositions and methods for the reduction of pain, such as neuropathic pain or inflammatory pain by administering to a subject a formulation comprising a purified combination of herbs comprising Scutellaria, Glycyrrhiza, Ziziphus and Paeonia.

A botanical formulation comprised of four different herbs has been used for the treatment of diarrhea, abdominal spasms, fever, headache, vomiting, nausea, extreme thirst, subcardial distention, and chemotherapy (U.S. Pat. No. 7,534,455; hereby incorporated by reference). The four herbs are Scutellaria, Glycyrrhiza, Ziziphus and Paeonia. Examples include Scutellaria baicalensis (from which the compound, Wogonin, is obtained), Glycyrrhiza uralensis, Ziziphus jujuba, and Paeonia lactiflora. Another exemplary combination comprises Scutellariae baicalensis Georgi (scute), Paeonia lactiflora pall (white peony root), Glycyrrhizae uralensis Fisch (licorice) and the fruit of Fructus ziziphi (date). Beneficial neurological effects are observed of Scutellaria, Glycyrrhiza, Ziziphus and Paeonia, and a combination of two, three, or four herbs listed above is useful to reduce the severity of pain as well as of neurological disorders. Such disorders include epilepsy/seizures, as well as disorders that respond to neuroprotective agents, e.g., these disorders include pain, including neuropathic pain, and injury to the brain or spinal cord, including traumatic and ischemic injuries.

Methods for preparing and administering the four-herb combination, e.g., PHY906, are described in U.S. Pat. No. 7,534,455, hereby incorporated by reference. The botanical combination drug PHY906 is manufactured by PhytoCeutica Inc. (New Haven, Conn.) pursuant to GMP standards and regulations and is in clinical trials for various clinical indications and uses (ClinicalTrials.gov Identifiers NCT00076609, NCT00036517, NCT00730158, and NCT 00411762. Methods of quantitively assessing the properties of botanical samples, e.g., for clinical use, is described in US Patent Publication No. 2005006572 as well as Australian Patent No. 779424, hereby incorporated by reference.

The four-herb combination comprises processed vegetative matter such as leaves, roots, or blossoms of the botanical species described above. For example, the composition includes aqueous solutions or tinctures (alcohol or other solvent-based) of vegetative matter or non-aqueous solutions. Alternatively, the composition comprises purified compounds (or a plurality of compounds) derived from the herb(s) or the chemically synthesized counterpart of the naturally-derived compound. The composition is administered to subjects as described above for Wogonin. For example, an aqueous herbal extract is administered orally.

The synergistic combination of the four-herb of the present invention demonstrates a significant utility in treating pain in a subject. When administered together, the composition shortens the onset time of being effective, and prolongs the effective duration. This four-herb combination provides a novel form of therapy for treating or reducing pain associated with any disease or condition, including but not limited to, postmastectomy pain syndrome, neuropathic pain, arthritis pain, neck pain, amputation stump pain, and pain due to skin tumor.

The present invention therefore provides compositions and methods for the reduction of pain by administering to a subject a combination of the four-herb. A combination of purified extract or compound or an analogue of four herbs is used to treat both acute and chronic pain indications. Accordingly, a method of preventing and reducing pain perception is carried out by administering to a subject a composition comprising purified combination of four herbs comprising Scutellaria, Glycyrrhiza, Ziziphus and Paeonia in an amount sufficient to reduce pain perception by at least 10% compared to the level of pain perception in the absence of the composition. Preferably, pain is reduced by 25%, 50%, 2-fold, 5-fold, 10-fold or more. Reduction in pain is measured using standard medical methods of evaluating and scoring pain, e.g., the McGill Pain Questionnaire.

The compositions are suitable for humans or other mammalian subjects, e.g., the mammal is a human, canine, feline, or equine subject. In one example, the subject to be treated is identified as suffering from neuropathic pain. For example, the subject is identified as suffering from diabetes, HIV infection, neuropathic pain related to chemotherapy, or pain associated with nerve compression such as sciatica or a herniated disc. Alternatively, the subject is identified as 310 suffering from inflammatory pain such as that associated with tissue injury, damage or disease.

Four herbs to be used in the methods described herein are purified, e.g., isolated from natural sources. A purified preparation of four herbs comprises at least 75%, 80%, 90% or 99%-100% extract by weight (w/w).

A method of reducing pain perception is characterized by identifying a subject with an injury and administering to the individual an amount of a combination of four herbs comprising Scutellaria, Glycyrrhiza, Ziziphus and Paeonia and/or Wogonin sufficient to reduce pain perception by at least 10% compared to the level of pain perception in the absence of a medicament. Preferably, perception of pain is reduced by at least 20%, 50%, 75%, eliminated or rendered imperceptible by the patient. In one example, the subject has been diagnosed with an injury to a bodily tissue or inflammation of a bodily tissue. For example, the injury is a cut/incision, abrasion, bruise, fracture, crush injury or is the result of a surgical procedure. The subject is identified as experiencing pain related to passage of kidney stones, a dental extraction, caesarian surgery, or cancer. For example, administration for pain is carried out using a sustained release formulation.

Alternatively, the subject is identified as experiencing pain in the absence of an injury or inflammation of a bodily tissue. A combination of four herbs comprising Scutellaria, Glycyrrhiza, Ziziphus and Paeonia and/or purified Wogonin is administered to the individual as described above in an amount sufficient to reduce pain perception by at least 10% compared to the level of pain perception in the absence of a medicament. Examples of such pain syndromes include patients identified as suffering from neuropathic pain, e.g., neuropathic pain associated with diabetes or HIV infection.

Isolation and Purification of Natural Product Compounds from Herbal Extracts

Substantially purified natural product compounds or “the compounds” of the present invention were prepared from herbal extracts. For example, the dry powder of the herbal plant (3 kg) were extracted in 80% aqueous MeOH (5 L) at room temperature overnight. Then, the filtrates were evaporated in vacuum under 40° C., and the concentrates were fractionized into three fractions (n-EtOAc, n-BuOH, and aqueous). Each solvent extract was flowed through column chromatography (SiO₂, ODS, Sephadex etc.) and eluted with several mixed solvents. Several fractions were repeatedly chromatographed while varying the packing materials or 340 eluting solutions. Purity of the compounds was assessed by TLC or HPLC. The molecular structure and weight were determined using one or more of the following techniques: 1) hypothesizing chemical and physical characteristics of compounds; 2) determining functional groups in the compound through IR spectra; 3) reading molecular weight and molecular formula from Mass data; 4) identifying chemical environment and integration value of proton, and neighboring protons using ¹H-NMR data; 5) measuring chemical environment and numbers of carbon from ¹³C-NMR data; 6) multiplicity of carbon by reading DEPT NMR data; 7) investigating the correlation between neighboring proton signals from ¹H-¹H 2D COSY NMR; 8) studying the correlation between carbon and neighboring proton signals using HSQC 2D NMR; 9) reading the long range correlations between carbon and proton signals from HMBC 2D NMR; 10) determining the stereochemistry of chiral carbons from NOESY 2D NMR; 11) determining the stereochemistry of a molecular structure by reading Polarimeter and CD; and 12) adaptating molecular modifications as needed (e.g., acetylation, methylation, methyl esterification, acid hydrolysis, alkaline hydrolysis).

### Pharmaceutical Compositions

The compositions described herein are suitable for pain reduction as well as the prevention or reduction in the severity and/or frequency of seizures. Compounds or extract are purified from the natural source, for example from the four herbs. Optionally, compounds or extract is formulated as mixtures of 4, 5, 8, 10 or more compounds. In some cases the compound or extract is formulated as a prodrug. The prodrug is inactive until it is ingested, 360 processed by the body (digested), or comes in contact with a target cell The prodrug chemical structure undergoes conversion to an active drug within a biological system, e.g., metabolism or contact with a component of a target cell such as an enzyme or cell surface structure. Some prodrugs include the active compound to which a chemical moiety has been linked, i.e., the compound has been derivatized. When the prodrug is metabolized, the chemical moiety is removed thereby activating the compound. Examples of targeted prodrug formulations include antibody-directed enzyme prodrugs, gene-directed enzyme prodrugs, and peptide transporter-associated prodrugs.

The purified compounds, extracts, and functional analogs of the present invention are administered by virtually any mode and are administered simultaneously or serially. When administered serially, the purified compounds and functional analogs are administered sufficiently close in time so as to provide the desired effect, for example within 1-3 hours of each other. In some examples, the purified compounds and functional analogs are administered topically, transdermally via a transdermal patch.

The purified compounds, extracts, and functional analogs are administered therapeutically to treat or reduce neropathic pain or inflammatory pain. For example, the purified compounds, extract, and functional analogs are administered to treat or alleviate the pain in a patient who is suffering from arthritis.

The purified compounds, extract, and functional analogs are administered to a subject, using a wide variety of routes or modes of administration. Suitable routes of administration for particular compositions include, but are not limited to, oral inhalation; nasal inhalation; transdermal; oral; rectal; transmucosal; intestinal; and parenteral administration, including intramuscular, subcutaneous, and intravenous injections. The purified compounds, extract, or functional analogs are administered by intrathecal and intraventricular modes of administration.

The purified compounds or extract from natural products such as herbal source are administered. In addition, the compounds or extract can be administered in a combination with other therapeutic agents. The choice of therapeutic agents that are co-administered with the composition of the invention will depend, in part, on the condition being treated. For example, the compounds of the invention are administered in cocktails comprising other agents used to treat symptoms and associated with pain. In the latter case, the combination therapy approach may permit a lower dose of the agents, thereby reducing undesired side effects.

The compounds or extract can be formulated either as single compound per se or as mixtures of compounds of the same type (e.g., two different analogs), as well as mixtures of compounds. Such compositions will generally comprise at least one purified compound or functional analogs formulated as a pharmaceutically acceptable salt or hydrate.

Pharmaceutical compositions for use in accordance with the present invention are formulated in conventional manner using one or more physiologically acceptable carriers, excipients, diluents or auxiliaries that further facilitate processing of the substantially purified natural product compounds. The choice of formulation is dependent upon the selected administration route. For example, the compounds are formulated in the form of an ointment, paste, spray, patch, cream, gel, sponge, or foam.

The formulations are administered by implantation or transcutaneous delivery (for example subcutaneously or intramuscularly), intramuscular injection, or transdermally. Thus, for example, the compounds are formulated with suitable polymeric or hydrophobic materials (such as an emulsion in acceptable oil) or ion exchange resins.

Formulations suitable for transdermal administration of compounds are described in U.S. Pat. Nos. 5,725,876; 5,716,635; 5,633,008; 5,603,947; 5,411,739; 5,364,630; 5,230,896; 5,004,610; 4,943,435; 4,908,213; and 4,839,174, which patents are hereby incorporated herein by reference. As purified compounds or functional analogs, pharmaceutically acceptable salts or hydrates are readily absorbed and cross cell membranes and the blood-brain barrier. Any of these formulations are routinely adapted for transdermal administration.

For injection, the purified compounds or functional analogs are formulated in physiologically compatible aqueous solutions, such as Hanks's solution, Ringer's solution, or 415 physiological saline buffer. For transmucosal administration, penetrants appropriate to the barrier to be permeated are used in the formulation. Such penetrants are generally known in the art.

For oral administration, the purified compounds or functional analogs are formulated with pharmaceutically acceptable carriers well known in the art. Such carriers enable the compounds of the invention to be formulated for oral administration as tablets, pills, gums dragees, capsules, liquids, gels, syrups, slurries, suspensions and the like. Alternatively, the compounds are formulated into candies, cookies, or other edible foodstuffs. Pharmaceutical preparations for oral use are obtained by mixing the compounds of the invention with a solid excipient, optionally grinding the resulting mixture, and processing the mixture of granules, after adding suitable auxiliaries, if desired, to obtain tablets or dragee cores. Suitable excipients include, but are not limited to, fillers, such as sugars, including lactose, sucrose, mannitol, or sorbitol; cellulose preparations, such as maize starch, wheat starch, rice starch, potato starch, gelatin, gum tragacanth, methyl cellulose, hydroxypropylmethyl-cellulose, sodium carboxymethylcellulose, and polyvinylpyrrolidone (PVP). If desired, disintegrating agents can be added, such as cross-linked polyvinyl pyrrolidone, agar, or alginic acid or a salt thereof, such as sodium alginate.

Concentrated sugar solutions are used that can optionally contain gum arabic, talc, polyvinyl pyrrolidone, carbopol gel, polyethylene glycol, or titanium dioxide, lacquer solutions, and suitable organic solvents or solvent mixtures. Dyestuffs or pigments are added to the tablets or coatings for identification or to characterize different combinations of active compound doses.

Pharmaceutical preparations that are used orally include push-fit capsules made of gelatin, as well as soft, sealed capsules made of gelatin and a plasticizer, such as glycerol or sorbitol. The push-fit capsules contain the compounds of the invention in an admixture with filler, such as lactose; binders, such as starches; or lubricants, such as talc or magnesium stearate; or stabilizers. In soft capsules, the compounds of the invention are dissolved or suspended in suitable liquids, such as fatty oils, liquid paraffin, or liquid polyethylene glycols. In addition, stabilizers are added to the soft-capsule formulation. All formulations for oral administration are in dosages suitable for such administration.

For buccal administration, the composition takes the form of oral sprays, tablets, gums, or lozenges formulated by well-known methods. A candy formulation suitable for oral or buccal administration of therapeutic compounds, pharmaceutically acceptable salts and hydrates is described in U.S. Pat. No. 6,083,962, which is hereby incorporated herein by reference. Additional formulations suitable for oral or buccal administration of therapeutic compounds, are described in U.S. Pat. Nos. 5,939,100; 5,799,633; 5,662,920; 5,603,947; 5,549,906; D358,683; 5,326,563; 5,293,883; 5,147,654; 5,035,252; 4,967,773; 4,907,606; 4,848,376; and 4,776,353, which are hereby incorporated herein by reference. All of these formulations are routinely adapted for administration of the purified compounds or functional analogs thereof, pharmaceutically acceptable salts and hydrates.

Compositions for inhalation or insufflation include solutions and suspensions in pharmaceutically acceptable, aqueous or organic solvents, or mixtures thereof, and powders. Preferably the compositions are administered by the oral or nasal respiratory route for local or systemic effect. For administration by oral or nasal inhalation, the compounds of the invention are conveniently delivered in the form of an aerosol spray delivered via pressurized packs or a nebulizer, with a suitable propellant, e.g., carbon dioxide or other suitable gas. In the case of a pressurized aerosol, the dosage unit is controlled by a dose-metered valve. Capsules and cartridges, e.g. gelatin, for use in an inhaler or insufflator are formulated as a powder mix of the compounds if the invention and a suitable powder base, such as lactose or starch. Formulations suitable for nasal inhalation are well known in the art. For example, a nasal aerosol spray contains compound, a water soluble diluent such as an organic acid, and a thickening agent such as a natural or synthetic polymer or an oil substance comprising the oil phase of an emulsion. The compounds of the invention are also administered in a vaporizer that delivers a volume of vapor containing compounds. The vaporizer is battery operated and designed to deliver a dosage of compound effective to reduce pain. The compounds of the invention, in a sterile pharmaceutically acceptable solvent, are nebulized by use of inert gases. Nebulized solutions are breathed directly from the nebulizing device or the nebulizing device is attached to a face mask, tent or intermittent positive pressure breathing machine.

In one embodiment, an aerosol spray containing substantially purified natural product compounds is used to treat or reduce pain symptoms.

For administration by injection, the compounds of the invention are formulated with a surface-active agent (or wetting agent or surfactant) or in the form of an emulsion (as a water-in-oil or oil-in-water, emulsion). Suitable surface-active agents include, but are not limited to, non-ionic agents, such as polyoxyethylenesorbitans (e.g. Tween™ 20, 40, 60, 80 or 85) and other sorbitans (e.g. Span™ 20, 40, 60, 80 or 85). Compositions with a surface-active agent comprise between 0.05 and 5% surface-active agent, and preferably between 0.1 and 2.5%. It will be appreciated that other ingredients are added, for example mannitol or other pharmaceutically acceptable vehicles, if necessary.

Suitable emulsions are prepared using commercially available fat emulsions, such as Intralipid™, Liposyn™, Infonutrol™, Lipofundin™ and Lipiphysan™. The active ingredient is either dissolved in a pre-mixed emulsion composition or alternatively it is dissolved in an oil (e.g. soybean oil, safflower oil, cottonseed oil, sesame oil. corn oil or almond oil) and an emulsion formed upon mixing with a phospholipid (e.g. egg phospholipids, soybean phospholipids or soybean lecithin) and water. It will be appreciated that other ingredients are added, for example gylcerol or glucose, to adjust the tonicity of the emulsion. Suitable emulsions typically contain up to 20% oil, for example, between 5 and 20%. The fat emulsion preferably comprises fat droplets between 0.1 and 1.0 μm, particularly 0.1 and 0.5 μm, and have a pH in the range of 5.5 to 8.0.

An injectable formulation containing compound or extract is used to treat or reduce pain in a subject. In such instances, the injectable formulation of compounds are provided in the form of a portable kit or package.

The purified compound or extract is formulated for parenteral administration by injection, e.g., by bolus injection or continuous infusion. Formulations for injection are presented in unit-dosage form, e.g., in ampules or in multi-dose containers, optionally with an added preservative. The compositions take such forms as suspensions, solutions, or emulsions in oily or aqueous vehicles, and contain formulatory agents, such as suspending, stabilizing, or dispersing agents.

Pharmaceutical formulations for parenteral administration include aqueous solutions of the compounds of the invention in water-soluble form. Additionally, suspensions of the compounds of the invention are prepared as appropriate oily-injection suspensions. Suitable lipophilic solvents or vehicles include fatty oils, such as sesame oil, or synthetic-fatty-acid esters, such as ethyl oleate or triglycerides, or liposomes. Aqueous-injection suspensions contain substances that increase the viscosity of the suspension, such as sodium carboxymethyl cellulose, sorbitol, or dextran. Optionally, the suspension contains suitable stabilizers or agents that increase the solubility of the compounds of the invention to allow for the preparation of highly concentrated solutions.

Alternatively, the compounds or extract is in powder form for constitution with a suitable vehicle, e.g., sterile pyrogen-free water, before use.

The compounds or extract can also be formulated in rectal compositions, such as suppositories or retention enemas, e.g., containing conventional suppository bases, such as cocoa butter or other glycerides.

The pharmaceutical compositions also comprise suitable solid- or gel-phase carriers or excipients. Examples of such carriers or excipients include, but are not limited to, calcium carbonate, calcium phosphate, various sugars, starches, cellulose derivatives, gelatin, and polymers, such as polyethylene glycols.

The appropriate dose of the pharmaceutical composition is that amount effective to prevent occurrence of the symptoms of the disorder or to treat some symptoms of the disorder from which the patient suffers. By “effective amount”, “therapeutic amount” or “effective dose” is meant that amount sufficient to elicit the desired pharmacological or therapeutic effects, thus resulting in effective treatment or alleviation of the disorder. The effective dose varies, depending upon factors such as the condition of the patient, the severity of the symptoms of the disorder, and the manner in which the pharmaceutical composition is administered. The effective dose of the composition differs from patient to patient but in general includes amounts starting where desired therapeutic effects occur, but below that amount where significant undesirable side effects are observed. Thus, when treating a seizures and epilepsy, an effective amount of composition is an amount sufficient to pass across the blood-brain barrier of the subject and to interact with relevant receptor sites in the brain of the subject and alter the actions of neurotransmitters on those receptors, thus resulting in effective prevention or treatment of the disorder.

### Administration and Dosages

Pharmaceutical preparations suitable for use with the present invention include compositions wherein the purified compounds, extract, or functional analogs are present in effective amounts, i.e., in amounts effective to achieve the intended purpose, for example, treating or reducing pain and seizures. Of course, the actual amounts of the compounds effective for a particular application depends upon a variety of factors including, inter alia, the type of disorder being treated, and the age and weight of the subject. When administered to treat or reduce pain, such compositions contain amounts of compound effective to achieve these results. Determination of effective amounts is well within the capabilities of those skilled in the art.

The compounds can be administered in any manner that achieves the requisite therapeutic or prophylactic effect. Therapeutically or prophylactically effective doses of the compounds of the invention can be determined from animal or human data for analogous compounds that are known to exhibit similar pharmacological activities. The applied doses are adjusted based on the relative bioavailability, potency and in vivo half-life of the administered compounds as compared with these other agents.

Adjusting the dose to achieve maximal efficacy in humans based on the methods described above and other methods that are well-known is well within the capabilities of the ordinarily skilled artisan.

Typically, the dosage range of natural product compounds administered subcutaneously, is about 0.1 mg/kg/day to 0.4 mg/kg/day. The dosage range of natural product compounds capsules for oral administration is in the range of about 100 μg/day to 2000 μg/day (See e.g., Ye et al (2000) Acta Pharmacol Sin: 21, 65; Ma et al. (1998) Ann NY Acad Sci. 854:506-7; and Ma et al. (1998) N-S Arch Pharmacol 358:suppl 1, P53194). Typically, the dosage range of compounds administered subcutaneously, is about 3 mg/kg/day to 12 mg/kg/day. The dosage range of substantially purified natural product compounds capsules for oral administration is in the range of about 3 mg/day to 60 mg/day.

It will be appreciated that the duration of action, and the therapeutic effect of the compounds is increased by combining two or more purified compounds or functional analogs of the present invention without causing adverse side effects. Furthermore, the dosage of each purified compound is reduced to achieve the same therapeutic effect. For the sake of illustration only, if a dosage of 2000 μg/day of one compound of the present invention is effective at treating seizures, but causes some undesired side effects, then the dosage of that natural product of the present invention may be reduced, for example to 1000 μg/day, when used in combination with a different natural product of the present invention, for example, at a dosage of about 30 mg/day. This combination provides the same therapeutic effect but without the adverse side effects. Furthermore, the duration of therapeutic effect may also increase by a using a combination compounds. As will be obvious to the skilled practitioner, the effective dosage amount can be manipulated to achieve the desired therapeutic effect.

The compounds of the invention are administered to subjects who suffer from pain. Regardless of the condition of the subject, the compounds of the invention are typically administered as part of a daily regimen.

The extract or compounds of the present invention can also be administered to the subject in a combination of a therapeutically effective amount of a secondary agent or a third agent. The secondary agent can be any pharmacologic agent known or suspected to be of benefit in the treatment or prevention of pain in a subject. For example, the secondary agent is a secondary pain relieving agent. Secondary pain relieving agents, which include morphine, are well-known to those of ordinary skill in the art. Examples of such agents include aspirin, acetaminophen (Tylenol) or other aspirin-like drugs called nonsteroidal anti-inflammatory drugs (NSAIDs), weak narcotics such as codeine (Tylenol with codeine), hydrocodone (Vicodin or Lortab), Percocet, Percodan or propoxyphene (Darvon), strong opioids such as morphine, Demerol, Dilaudid, fentanyl (duragesic patches) and methadone.

The ratio between toxicity and therapeutic effect for a particular compound is its therapeutic index and is expressed as the ratio between LD₅₀ (the amount of compound lethal in 50% of the population) and ED₅₀ (the amount of compound effective in 50% of the population). The compounds of the invention that exhibit high therapeutic indices are preferred. Therapeutic index data is obtained from animal studies and used in formulating a range of dosages for use in humans. The dosage of such compounds preferably lies within a range of plasma concentrations that include the ED₅₀ with little or no toxicity. The dosage varies within this range depending upon the dosage form employed and the route of administration utilized.

The herb combination composition is useful for preventing, reducing the severity, frequency, or duration of a seizure. For example, the patient to be treated identified as suffering from or at risk of developing a seizure disorder, e.g., the subject is diagnosed with epilepsy or is diagnosed with both epilepsy and alzheimer's disease (AD) (e.g., the subject has a score of less than 27 on a Mini Mental State Examination (MMSE)).

The term “seizure” as used herein refers to a change in behavior, or spasms or convulsions that arise naturally in a subject as a result of a natural chemical imbalance or lack of homeostasis in a subject. Such natural convulsions may arise due to a disease or disorder (e.g., epilepsy), age, or the occurrence of an event (e.g., stroke). The term “seizure” also refers to seizures that are chemically induced, for example those brought on by intake, uptake, or ingestion of chemicals such as organophosphates.

The term “antiepileptogenic” refers to inhibiting at least one of the processes that underlie the development of epilepsy.

### Reduction in Seizure Disorders

Two to 4 million Americans suffer from recurrent seizures. Methods of diagnosing individuals suffering from or at risk of developing a seizure disorder are well known in the art (Saunders Manual of Neurologic Practice, R. Evans, ed. p. 244-255; Office Practice of Neurology, 2^{nd} edition, Samuels et al., eds. Churchill, Livingston Press, p. 928-937).

The herb combinations or combinations of compounds obtained from the two or more of the four listed herbal sources are useful to treat a diverse range of seizures or preventing epilepsy and the onset of seizures (epileptogenesis). Seizures are typically divided into generalized seizures (absence, atonic, tonic-clonic, myoclonic) and partial (simple and complex) seizures.

A significant advantage of the herbal combination composition compared to other drugs used for epilepsy is that these herbs or compounds derived therefrom not only prevent and reduce the frequency/severity/duration of epileptic seizures, these compounds also have the added benefit of neuroprotection by virtue of their interaction with acetylcholinesterase. Epilepsy, particularly temporal lobe epilepsy, can be associated with progressive memory deterioration (by a mechanism unrelated to AD) and becomes more severe with ongoing seizures. The herbal combination composition reduces or slows the progression of epilepsy-associated memory loss.

### (a) Generalized Seizures

Generalized seizures affect both cerebral hemispheres (sides of the brain) from the beginning of the seizure. They produce loss of consciousness, either briefly or for a longer period of time, and are sub-categorized into several major types: generalized tonic clonic; myoclonic; absence; and atonic.

Absence seizures (also called petit mal seizures) are lapses of awareness, sometimes with staring, that begin and end abruptly, lasting only a few seconds. There is no warning and no after-effect. Some absence seizures are accompanied by brief myoclonic jerking of the eyelids or facial muscles, or by variable loss of muscle tone. More prolonged attacks may be accompanied by automatisms, which may lead them to be confused with complex partial seizures. However, 635 complex partial seizures last longer, may be preceded by an aura, and are usually marked by some type of confusion following the seizure.

Myoclonic seizures are rapid, brief contractions of bodily muscles, which usually occur at the same time on both sides of the body. Occasionally, they involve one arm or a foot. People usually think of them as sudden jerks or clumsiness. A variant of the experience, common to many people who do not have epilepsy, is the sudden jerk of a foot or limb during sleep.

Atonic seizures produce an abrupt loss of muscle tone. Other names for this type of seizure include drop attacks, astatic or akinetic seizures. They produce head drops, loss of posture, or sudden collapse. Because they are so abrupt, without any warning, and because the people who experience them fall with force, atonic seizures can result in injuries, such as to the head and face.

Generalized tonic clonic seizures (grand mal seizures) are the most common and best known type of generalized seizure. They begin with stiffening of the limbs (the tonic phase), followed by jerking of the limbs and face (the clonic phase). During the tonic phase, breathing may decrease or cease altogether, producing cyanosis (blueing) of the lips, nail beds, and face. Breathing typically returns during the clonic (jerking) phase, but it may be irregular. This clonic phase usually lasts less than a minute. Some people experience only the tonic, or stiffening phase of the seizure; others exhibit only the clonic or jerking movements; still others may have a tonic-clonic-tonic pattern.

### (b) Partial Seizures

In partial seizures the onset of the electrical disturbance is limited to a specific area of one cerebral hemisphere (side of the brain). Partial seizures are subdivided into simple partial seizures (in which consciousness is retained); and complex partial seizures (in which consciousness is impaired or lost). Partial seizures may spread to cause a generalized seizure, in which case the classification category is partial seizures secondarily generalized.

Partial seizures are the most common type of seizure experienced by people with epilepsy. Virtually any movement, sensory, or emotional symptom can occur as part of a partial seizure, including complex visual or auditory hallucinations. There are two types of partial seizure, simple partial seizures and complex partial seizures.

Complex partial seizures generally affect a larger area of the brain than simple partial seizures and they affect consciousness. During a complex partial seizure, a person cannot interact typically normally with other people, is not in control of his movements, speech, or actions; doesn't know what he's doing; and cannot remember afterwards what happened during the seizure. Although someone may appear to be conscious because he stays on his feet, his eyes are open and he can move about, it will be an altered consciousness, a dreamlike, almost trancelike state. A person may even be able to speak, but the words are unlikely to make sense and he or she will not be able to respond to others in an appropriate way. Although complex partial seizures can affect any area of the brain, they often affect one or both of the brain's two temporal lobes. Because of this, the condition is sometimes called “temporal lobe epilepsy.”

### (c) Epilepsy

Epileptic seizures are the outward manifestation of excessive and/or hypersynchronous abnormal activity of neurons in the cerebral cortex. Many types of seizures occur, as described above. The neuromechanism responsible for seizures includes the amygdala, the hippocampus, the hypothalamus, the parolfactory cortex, the frontal and temporal lobes, and the involvement of the substantia nigra, a particular portion of the brain considered to be part of neural circuitry referred to as the basal ganglia.

The methods and compositions of the invention are to be used to inhibit, reduce, or treat seizures that include, but are not limited to, tonic seizures, tonic-clonic seizures, atypical absence seizures, atonic seizures, myoclonic seizures, clonic seizures, simple partial seizures, complex partial seizures, and secondary generalized seizures.

Various in vivo models of seizures and epilepsy are used to test compositions against specific forms of seizures and epilepsy, e.g., Maximal Electroshock (MES) model and Subcutaneous Metrazole (SCMET) model. For example, the kainate model is an epileptic model in which kainic acid, one of the excitatory amino acids found in the brain, is injected to nuclei (amygdala, hippocampus, etc.) in the limbic system in a microamount to induce focal epilepsy. The kainate model serves as a model for an epileptic seizure; more particularly, as a model for status epilepticus induced from the limbic system in an acute phase, and as a model for evolution of a spontaneous limbic seizure to a secondary generalized seizure in a chronic phase. The kainate model may also be used as a cortex epilepsy model through injection of kainic acid to the cortex (sensory motor field). For a review of animal models used for epilepsy and seizures, see for example, Sarkisian (2001) Epilepsy and Behavior, 2: 201-216).

To investigate the neurotoxicity levels of the compositions, the Toxicity Model (TOX) using a rotorod is employed. The animals (e.g., mice) were trained to stand on an accelerating rotorod rotating at 10 rev min⁻¹ with a diameter of 3.2 cm. The trained animal is given the composition at various doses and the effect of the composition on their motor skills is determined. The dose at which the animals fell off the rotorod is the toxic dose.

### (d) Epileptogenesis

Epileptogenesis is the process by which a normal brain becomes chronically prone to seizures. Many brain insults (stroke, trauma, neurodegenerative disease etc) can induce epileptogenesis, yet no therapies exist to disrupt this process. Although reorganization of specific neuronal circuits and alterations in individual synapses are associated with epileptogenesis, the functional consequences and relative importance of these changes to epileptogenesis and seizure genesis are unknown, as are many of the molecular and cellular mechanisms underlying these alterations.

Seizures and epilepsy are common sequelae of acute brain insults such as stroke, traumatic brain injury, and central nervous system infections. Early, or acute symptomatic, seizures occur at the time of the brain insult and may be a marker of severity of injury. A cascade of morphologic and biologic changes in the injured area over months to years leads to hyperexcitability and epileptogenesis. After a variable latency period, late unprovoked seizures and epilepsy occur. The drugs that presently used in the treatment of epilepsy, treat the symptom, seizures, but do not modify the epileptogenic process.

The composition is administered during the latent period for the prevention of epileptogenesis and the development of unprovoked seizures and epilepsy, and the herb combination composition is used as a neuroprotectant and an antiepileptogenic agent.

### Wogonin for Pain Alleviation

Wogonin (Wog; 5,7-dihydroxy-8-methoxyflavone; CAS Registry Number 632-85-9) is a flavonoid derived from the root of Scutellaria baicalensis Georgi, a Chinese herbal remedy widely used in clinical treatment of inflammatory diseases, including atopic dermatitis, hyperlipemia, and atherosclerosis.

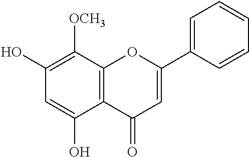 [SMILES: COc1c(O)cc(O)c2c(=O)cc(-c3ccccc3)oc12]

Wogonin and variations or analogues thereof have been described, e.g., in Gurung S K, Kim H P, Park H. Inhibition of prostaglandin E2 production by synthetic Wogonin analogs. Arch Pharm Res. 2009 Nov; 32(11):1503-8.

Isolation and identification of Flavonoids From the Roots of Scutellaria baicalensis

Plant materials (Scutellaria baicalensis) were obtained and purified (FIG. 3). The dried and powdered roots of S. baicalensis (3.4 kg) were extracted three times at room temperature with 80% aqueous MeOH (18 L×3). The extracts were partitioned with water (4 L, 972 g), EtOAc (4 L×2, 62g) and n-BuOH (4 L×2, 56 g), successively. The EtOAc extract (62 g) concentrated under vacuum was dissolved in CHCl₃ and MeOH and precipitated for a day. The precipitate was filtered to yield compound 2 (SBE-D, baicalein, 14 g). The concentrate of the filtrate in vacuo (48 g) was applied to the silica gel column chromatography (c.c.) (9×20 cm) and eluted with CHCl₃:MeOH (30:1→20:1→10:1) and monitored by thin layer chromatography (TLC) to produce 33 fractions (SBE-1 to SBE-33). The precipitates of SBE-7˜SBE14 fractions were filtered, washed and concentrated to produce compound 3 (SBE-7D, 740 Wogonin, 2.3g).

Instrumentation Column chromatography was performed on silica gel 60 (63˜200 μm).

1H-NMR (400 MHz) and 13C-NMR (100 MHz) spectra were obtained using Varian Unity Inova AS 400 FT-NMR spectrometer.

(Wogonin): 1H-NMR (400 MHz, DMSO, δ): 8.07 (2H, dd, J=7.6, 2.8 Hz, H-2′, 6′), 7.50 (3H, m, H-3′, 4′, 5′), 7.02 (1H, s, H-3), 6.79 (1H, s, H-6). 13C-NMR (100 MHz, DMSO, δ): 182.15 (C-4), 162.86 (C-2), 158.47 (C-7), 157.44 (C-5), 149.88 (C-9), 131.48 (C-4′), 131.28 (C-1′), 129.88 (C-3′, 5′), 128.73 (C-8), 126.10 (C-2′, 6′), 105.46 (C-3), 104.37 (C-10), 99.91 (C-6), 60.80 (OCH3).

### Formulations

Wogonin is purified and formulated for human use. A composition containing a purified Wogonin is optionally formulated in a sustained release delivery vehicle. The vehicle includes dermal patch, an intravenous pump, or another implantable device. The implant is inert, biodegradable, or erodible. For sustained release in an implant, patch, or oral composition, the vehicle contains a semipermeable membrane. The membrane serves the purpose of controlling the rate of delivery of Wogonin to bodily tissues. In another example of a sustained release formulation, the vehicle contains a plurality of particles, each of which are characterized as having a different rate of dissolution. For example, a composition may contain two or more classes of particles: slow, medium, and rapid release particles. A dosing regimen contains one or more doses of a sustained release formulation as needed to manage pain, e.g., reduce the frequency, severity, or duration of pain. Alternatively, one or more high doses (5, 10, 15, 20, or 30 mg/kg/day) are administered followed by lower doses (0.1-5 mg/kg/day) for management of symptoms. Other formulations include an ointment, paste, spray, patch, cream, gel, resorbable sponge, foam, or subcutaneous depo formulation. Administration is prior to, during, or after the onset of pain.

Wogonin is also useful as a neuroprotective agent in the context of patient undergoing surgery. For example, the patient is administered Wogonin for a period of time prior to an operation (e.g., 24, 12, 8, 4, 2, 1 hour prior to surgery). Optionally, administration is continued for a period of time after surgery (e.g., 1, 2, 4, 8, 12, 24, 48, 96 hours after surgery) or until cognitive difficulties diminish or are eliminated.

### Neuroprotective Activity

Studies were carried out to determine whether application of Wogonin can prevent N-methyl-D-Aspartate receptor (NMDAR)-mediated excitotoxicity in primary neuronal cultures from mouse cortex in vitro. In particular, detailed dose/response curves of Wogonin applied together with and for the duration of application of NMDA were generated. NMDAR-induced cytotoxicity can lead to fulminant cell death, necrosis, or a delayed form of cell death (apoptosis). While necrosis leads to immediate cell lysis, apoptotic cell death develops over time and is distinguished by a number of criteria such as characteristic morphological changes and DNA fragmentation. The LDH assay was used for the determination of cell death. This assay assesses primary necrosis as well as secondary, necrotic cell death subsequent to apoptosis. The results showed that Wogonin reduced the numbers of neurons that died when exposed to NMDA, thereby indicating that Wogonin has a neuroprotective effect.

For these experiments, cytotoxicity assays were conducted in primary cultures of mouse cortical neurons after 10 days in vitro using an art-recognized assay systema (Sun X, Chan L N, Gong X, Sucher N J. N-methyl-D-Aspartate receptor antagonist activity in traditional Chinese stroke medicines. NeuroSignals, 2003; 12:31-38.) Cultured neurons were exposed to NMDA (200 μM; 5 minutes) and co-agonist glycine (10 μM) in the nominal absence of magnesium and the presence of increasing concentrations of Wogonin. Measurements were performed for each dose of Wogonin in triplicate in at least three independent experiments. Wogonin was added to the culture in three different ways: 1) before, concomitant with and after NMDA, 2) concomitant with and after NMDA and 3) after NMDA. Twenty-four hours after exposure to NMDA cell death is determined by colorimetric lactate dehydrogenase (LDH) assay.

FIG. 2 shows the dose-dependent neuroprotective effect of Wogonin in this experimental model ranging from 0% protection at a dose of 10⁻⁹ M to >75% at 10⁻⁴ M. These results indicate that Wogonin inhibited/blocked neuronal death when the neurons were exposed to N-methyl-D-aspartate (IC₅₀ =2 μM).

### Wogonin Alleviates Pain

Purified Wogonin was tested in a standard mouse formalin pain model and was found to have anti-nociceptive activity in the mouse formalin pain model. The formalin test for antinociceptive activity is described in numerous publications, e.g., Hunskaar, S. O. B. Fasmer, and K. Hole, J. Neurosci. Methods 14: 69-76 (1985). The formalin test is a chemically-induced tonic pain model in which biphasic changes of nociceptive behavior are assessed and spinal/supraspinal plasticity of nociception is considered as a molecular basis for neuropathic pain particularly during the second (late) phase of the test, during which most clinically used drugs against neuropathic pain are active. The formalin test is accepted as an art recognized model of persistent clinical pain.

The formalin test (Wheeler-Aceto H, Porreca F, Cowan A. The rat paw formalin test: comparison of noxious agents. Pain 1990; 40:229-38) involves injection of 0.5% formalin into the mouse hind paw. The injection elicits a distinct biphasic behavioral profile in response to the formalin injection characterized by licking of the affected paw, and the number of licks is measured as a proxy for perceived pain. The first (acute) phase corresponds to the direct stimulation of peripheral fibers, while the second (inflammatory) phase is caused by the release of inflammatory mediators from the damaged tissue and nerve endings. Thus, this model is ideally suited to test the efficacy of Wogonin against the acute and chronic hyper-responsive neuronal discharges following activation of peripheral nerve fibers.

The results of the formalin test are given both as raw data (“Response”) and in an aggregate, analyzed form (“Analysis”). In the Response section, each Trial involves sixteen animals, eight controls given an i.p. injection of vehicle and eight given Wogonin at a specified dose. The data recorded for each animal is the amount of time spent licking the affected hind paw in a two-minute period. These two-minute periods occur at five-minute intervals and continue for 45 minutes. Plotting the time spent licking versus time reveals the characteristic biphasic response. From this plot, the area under the cure (AUC) is determined for each animal during both the acute and inflammatory stages. The AUC for each phase is shown in the analysis section of the data sheet for both control and Wogonin-treated animals. The AUC for each Wogonin-treated animal is compared to the average result from the control group, yielding an average percent of control (reported with the SEM and p value). Significant reductions in this number indicate a reduction in licking, and, presumably, a reduction of perceived pain.

A significant response to both the acute and the inflammatory phases of the test was observed with Wogonin at 200 mg/kg given intraperitoneally in the mouse formalin test. As can be seen in the Analysis section of the below table, there was an average 72.3% control in the acute phase and an average 74.9% control in the inflammatory phase. Thus there was significant reduction in licking in Wogonin-treated animals in both phases of the formalin test compared to controls indicating a significant reduction in licking, and, presumably, a reduction of perceived pain. Overt toxicity was not seen in the mouse models.

Male Swiss Webster NIH mice (20-30 g; Harlan, San Diego, Calif.) were used in the experiments described herein. Food was withdrawn on the day of experiment. Mice were placed in Plexiglass jars for at least 1 hour to accommodate to the environment. Following the accommodation period, mice were weighed and given Wogonin administered intraperitoneal (i.p.) or the appropriate volume of vehicle (10% Tween-80). Fifteen minutes after the i.p. dosing, mice were injected with formalin (20 μL of 5% formaldehyde solution in saline) into the dorsal surface of the right hind paw. Mice were transferred to the Plexiglass jars and monitored for the amount of time spent licking or biting the injected paw. Periods of licking and biting were recorded in 5 minute intervals for 40 minutes after the formalin injection. All experiments were done in a blinded manner. The early phase (acute) of the formalin response is measured as licking/biting between 0-10 minutes, and the late phase (inflammatory) is measured from 10-40 minutes. Differences between vehicle and Wogonin-treated groups are analyzed by one-way analysis of variance (ANOVA). A P value <0.05 is considered significant. Activity in blocking the acute and second phase of formalin-induced paw-licking activity is indicative that compounds are efficacious for acute and chronic pain. A significant response to both the acute and the inflammatory phases of the test was observed.

These results tabulated below indicate that Wogonin is useful as a pain medication.

Analysis

Area Under the Curve

| Dose | | | Drug | % of | | |
|---|---|---|---|---|---|---|
| (mg/kg) | Test | Control | Treated | control | S.E.M | p Value |
| 200 | Acute | 211.81 | 58.7 | 27.7 | 9.0 | <0.01 |
| 200 | Inflammatory | 583.7 | 146.3 | 25.1 | 9.3 | z 0.01 |

### Response

Duration of Licking (sec)

| Dose | Animal | | | | | | | | | |
|---|---|---|---|---|---|---|---|---|---|---|
| Mg/kg | # | 0 min | 5 min | 10 min | 15 min | 20 min | 25 min | 30 min | 35 min | 40 min |
| 0 | 01 | 65.02 | 0.00 | 0.00 | 0.00 | 11.45 | 25.47 | 30.68 | 81.58 | 0.00 |
| 0 | 02 | 46.78 | 37.09 | 0.00 | 0.00 | 0.00 | 0.00 | 45.11 | 0.00 | 0.00 |
| 0 | 03 | 55.16 | 10.07 | 0.00 | 0.00 | 22.14 | 37.97 | 40.09 | 0.00 | 2.17 |
| 0 | 04 | 69.70 | 0.00 | 0.00 | 0.00 | 10.40 | 45.39 | 48.66 | 27.53 | 2.18 |
| 0 | 05 | 75.87 | 3.01 | 0.00 | 0.00 | 19.01 | 64.70 | 20.80 | 0.00 | 94.32 |
| 0 | 06 | 55.98 | 27.96 | 0.00 | 0.00 | 46.32 | 65.30 | 40.69 | 0.00 | 0.00 |
| 0 | 07 | 85.46 | 0.00 | 0.00 | 5.53 | 24.85 | 0.00 | 10.16 | 59.12 | 0.00 |
| 0 | 08 | 67.64 | 0.00 | 0.00 | 20.66 | 36.13 | 36.38 | 21.54 | 0.00 | 0.00 |
| Dose | | | | | | | | | | |
| Mg/kg | Animal | 0 mm | 5 min | 10 min | 5 min | 20 min | 25 min | 30 min | 35 min | 40 min |
| 200 | 01 | 0.00 | 0.00 | 0.00 | 0.00 | 0.00 | 0.00 | 0.00 | 0.00 | 0.00 |
| 200 | 02 | 0.00 | 0.00 | 0.00 | 0.00 | 0.00 | 3.89 | 0.00 | 0.00 | 0.00 |
| 200 | 03 | 0.00 | 0.00 | 0.00 | 0.00 | 0.00 | 0.00 | 0.00 | 0.00 | 0.00 |
| 200 | 04 | 35.48 | 0.00 | 0.00 | 0.00 | 0.00 | 0.00 | 29.12 | 21.24 | 20.18 |
| 200 | 05 | 21.75 | 0.00 | 0.00 | 0.00 | 0.00 | 0.00 | 55.07 | 0.00 | 27.36 |
| 200 | 06 | 53.96 | 0.00 | 0.00 | 0.00 | 16.00 | 20.61 | 2.95 | 12.73 | 0.00 |
| 200 | 07 | 44.26 | 0.00 | 0.00 | 0.00 | 0.00 | 10.96 | 37.69 | 0.00 | 0.00 |
| 200 | 08 | 32.29 | 0.00 | 0.00 | 0.00 | 0.00 | 0.00 | 0.00 | 0.00 | 0.00 |

Other embodiments and uses of the invention will be apparent to those skilled in the art from consideration of the specification and practice of the invention disclosed herein. All U.S. Patents, scientific journal articles, and other references noted herein for whatever reason are specifically incorporated by reference. The specification and examples should be considered exemplary only with the true scope and spirit of the invention indicated by the following claims.

The invention is illustrated by way of working examples that demonstrate the anticonvulsive effect of the substantially purified natural product compounds of the invention in mice and rats.

### EXAMPLE

### Effect of Herbal Extracts in Mice and Rats

The herbal extracts were administered in an i.p. form, soluble form (SOL) or as a suspension (SUS). To produce a suspension, the herbal extract can be ground to a powder using a mortar and pestle, and the powder mixed with methyl cellulose. This mixture was then sonicated to produce a suspension that was subsequently administered to mice or rats. Various time points were analyzed at each concentration of herbal extract with each animal model and the results were recorded.

Studies were also conducted to determine the time course of herbal extracts in mice and rats following i.p. or oral administration of the extracts. The herbal extracts were prepared as described above at various concentrations. The effects were tested in the MES, ScMET, TOX and 6-Hz animal models.

### Effect of TJ-960 Extract in Mouse

TJ-960 (mixture of extracts of Bupleuri Radix, Paeoniae Radix, Pinelliae Tuber, Cinnamomi Cortex, Zizyphi Fructus, Scutellariae Radix, Ginseng Radix, Glycyrrhizae Radix, Zingiberis Rhizoma) was found to be active in the mouse-i.p. 6 Hz model. The TJ-960 extract was active in the 6-Hz model with an ED₅₀ of 36.4 mg/kg. In the mouse-i.p. toxicity model, 300 mg/kg is non-toxic. The lowest anti-convulsant dose is 15 mg/kg in the mouse-i.p. model and 30 mg/kg in the rat-i.p. model.

### Effect of TJ-10 Extract in Mouse and Rat and Effect of TJ-10 Extract in Rat Oral

TJ-10 (mixture of extracts of Bupleurum falcatum (thorowax) root, 19%; Peonia lactiflora (peony) root, 19%; Pinellia Ternata (ban xia) rhizome, 15%; Zizyphus jujuba (jubube) fruit, 12%; Panax ginseng (Asian ginseng) root, 9%; Scutellaria baicalensis (Skullcap) root, 9%; Zingiber officinale (ginger) rhizome, 6%; Cinnamomum cassia (cassia) bark, 6%; Glycyrrhiza uralensis (licorice, gan cao) rhizome, 5%) was active in ScMET rat-i.p. and rat-oral models and 6-Hz mouse-i.p. The lowest anticonvulsant dose was 100 mg/kg in the mouse-i.p. 6-Hz model and doses up to 300 mg/kg are non-toxic, including a 250 mg/kg dose given qd×3 days.

### TJ-12 Extract in Rat and Mouse

TJ-12 extract was found to be active in the MES rat-i.p. model.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

The patent and scientific literature referred to herein establishes the knowledge that is available to those with skill in the art. All United States patents and published or unpublished United States patent applications cited herein are incorporated by reference. All published foreign patents, patent applications, and other references cited herein are hereby incorporated by reference. Genbank and NCBI submissions indicated by accession number cited herein are hereby incorporated by reference.

While this invention has been particularly shown and described with references to preferred embodiments thereof, it will be understood by those skilled in the art that various changes in form and details may be made therein without departing from the scope of the invention encompassed by the appended claims.

What is claimed is:

1. A pharmaceutical composition comprising a purified compound or processed plant material of a combination of two to four herbs selected from the group consisting of Scutellaria, Glycyrrhiza, Ziziphus and Paeonia, or extracts thereof.

2. The composition of claim 1, wherein said composition comprises an extract or purified compound of said herbs.

3. The composition of claim 1, further comprising Wogonin.

4. A method of preventing and reducing pain perception, comprising administering to a mammal a composition comprising a combination of two to four herbs selected from the group consisting of Scutellaria, Glycyrrhiza, Ziziphus and Paeonia, or extracts thereof, wherein said composition reduces pain perception by at least 10% compared to the level of pain perception in the absence of said composition.

5. The method of claim 4, wherein said composition comprises an extract or purified compound of each of said herbs.

6. The method of claim 4, further comprising administering Wogonin.

7. A method of preventing the onset of and reducing the severity of a seizure disorder, comprising administering to a mammal a composition comprising a combination of two to four herbs selected from the group consisting of Scutellaria, Glycyrrhiza, Ziziphus and Paeonia, or extracts thereof, wherein said composition prevents the onset of or the severity of a seizure disorder.

8. The method of claim 7, wherein said composition comprises an extract or purified compound of said herbs.

9. The method of claim 7, further comprising administering Wogonin.

10. A method of preventing and reducing pain perception, comprising administering to a mammal a composition comprising purified Wogonin in an amount sufficient to reduce pain perception by at least 10% compared to the level of pain perception in the absence of said composition.

11. The method of claim 10, wherein said mammal is a human, canine, feline, or equine subject.

12. The method of claim 10, wherein said mammal is identified as suffering from neuropathic pain.

13. The method of claim 10, wherein said mammal is identified as suffering from tissue injury, damage or disease.

14. The method of claim 10, wherein said mammal is identified as suffering from diabetes.

15. The method of claim 10, wherein said mammal is identified as comprising an HIV infection.

16. The method of claim 10, wherein said mammal is identified as suffering from neuropathic pain related to chemotherapy.

17. The method of claim 10, wherein said purified Wogonin comprises at least 99% Wogonin by weight.

18. The method of claim 10, wherein said Wogonin is administered at a dose of 0.1 mg/kg/day to 1000 mg/kg/day.

19. The method of claim 10, wherein said composition is administered in a sustained release delivery vehicle.

20. The method of claim 10, wherein said composition comprises an adhesive dermal patch.

21. The method of claim 10, wherein said composition is continuously infused into said subject.

22. The method of claim 10, wherein said composition is administered to said subject by an intravenous pump.

23. The method of claim 10, wherein said composition is orally ingested.

24. The method of claim 10, wherein said composition comprises an implant, said implant being biodegradable or erodible.

25. The method of claim 10, wherein said composition comprises a semipermeable membrane, said membrane being delivery rate controlling.

26. The method of claim 10, wherein said composition comprises a plurality particle, each of said particles comprising a different rate of dissolution.

27. The method of claim 10, wherein said composition is administered in the form of an ointment, paste, spray, patch, cream, gel, sponge, foam, or subcutaneous depo formulation.

28. A pharmaceutical composition comprising purified Wogonin in an amount to reduce pain perception.
